(19) United States
(12) Patent Application Publication
(10) Pub. No.: US 2009/0155359 A1
(43) Pub. Date: Jun. 18, 2009

(54) GRANULATED PARTICLES, TABLETS AND METHOD FOR PRODUCING GRANULATED PARTICLES

(75) Inventors: Teruo Nishito, Naka-gun (JP); Tomiyuki Yanase, Yamato-shi (JP)

Correspondence Address: MERCHANT & GOULD PC, P.O. BOX 2903, MINNEAPOLIS, MN, 55402-0903, US

(73) Assignee: LION CORPORATION, Sumida-ku (JP)

(21) Appl. No.: 12/298,438

(22) PCT Filed: Apr. 27, 2007
(86) PCT No.: PCT/JP2007/059193

§ 371 Date: Oct. 24, 2008

(30) Foreign Application Priority Data
Apr. 28, 2006 (JP) 2006-126384

(51) Int. Cl. A61K 9/20; A61K 9/14; A61P 43/00

(52) U.S. Cl. 424/464; 424/489

(57) ABSTRACT

The present invention relates to granulated particles including a poorly water soluble drug (A) and a diluent (B) and characterized in that a volume average particle size of the particles of the poorly water soluble drug (A) and the particles of the diluent (B) is within the range of 0.01 to 35 μm and a water soluble- or water swellable polymer compound (C) having a viscosity of less than 6.0 mPa·s in an aqueous solution thereof of 2% by mass at 20° C. is further contained, a tablet containing the granulated particles, and a method for producing the granulated particles.

### TECHNICAL FIELD

The present invention relates to granulated particles, a tablet, and a method for producing granulated particles.

Priority is claimed on Japanese Patent Application No. 2006-126384, filed Apr. 28, 2006, the content of which is incorporated herein by reference.

### BACKGROUND ART

Solid chemical compositions are used in the form of tablets, capsules, granulated agents, or the like depending on the intended use.

Using an active ingredient (drug) as well as an adequate diluent as source materials, the aforementioned granulated agents are prepared by producing particles due to, for example, a dry granulation process involving a roll compressor or a wet granulation process involving an extrusion granulator, or a wet granulation process in which an active ingredient (drug) is treated with a water-soluble polymer compound by employing a fluidized-bed granulating machine, and then granulating the produced particles.

In addition, the tablets are produced by, for example, further adding an additive (tablet material) to a granulated agent or a granular composition obtained as described above and then tabletting the resulting mixture. Moreover, hard capsules, divided powders, and the like are obtained by filling a certain amount of the aforementioned granulated agent in a packaging container or the like.

When preparing a solid chemical composition, various attempts for the formulation have been made in many cases during the production of granulated particles in order to generally improve drug absorption in the body or administerability.

Among these attempts, when the drug contained in the granulated particles is a poorly water soluble drug having low water solubility, the poorly water soluble drug is hardly absorbed in the body, and especially when the drug is crystalline or has a large particle size, it is necessary to further refine the formulation process in order to secure satisfactory drug release properties in the body.

It should be noted that in the present description, the phrase “drug release properties in the body” refers to the degree of drug release when the granulated particles or the tablets containing the granulated particles are administered orally and the drug contained in the granulated particles is released in the oral cavity (in the body) from the granulated particles.

In order to secure satisfactory drug release properties in the body, a method to grind a poorly water soluble drug so as to increase the surface area of the poorly water soluble drug (refer to Patent Document 1) or a method to use an organic solvent has been proposed in the past.

In addition, a method to granulate the ground product of a poorly water soluble drug has also been proposed.

[Patent Document 1] Published Japanese translation No. 2004-530558 of PCT International Publication

### DISCLOSURE OF INVENTION

### Problems to be Solved by the Invention

In the method disclosed in Patent Document 1, some poorly water soluble drugs attach to the grinder when grinding the poorly water soluble drugs, depending on the types of poorly water soluble drugs, resulting in the considerable decline in the grindability thereof. Accordingly, it is impossible to secure satisfactory drug release properties in the body in these cases.

Moreover, in the method using an organic solvent, depending on the types of poorly water soluble drugs, some of them hardly dissolve in the organic solvent or the drug release properties in the body are not satisfactory in some cases.

Furthermore, in the method to granulate the ground product of a poorly water soluble drug, the drug release properties in the body decline in some cases, depending on the types of binders (polymer compounds or the like) to be used.

The present invention is made in view of the above circumstances and its object is to provide granulated particles excellent in terms of the drug release properties in the body, a tablet, and a method for producing the granulated particles.

### Means for Solving the Problems

As a result of intensive studies in order to solve the abovementioned problems, the present inventors completed the present invention.

That is, a first aspect according to the present invention is granulated particles including a poorly water soluble drug (A) and a diluent (B) and characterized in that a volume average particle size of the particles of the poorly water soluble drug (A) and the particles of the diluent (B) is within the range of 0.01 to 35 μm and a water soluble- or water swellable polymer compound (C) having a viscosity of less than 6.0 mPa·s in an aqueous solution thereof of 2% by mass at 20° C. is further contained.

In addition, it is preferable that the granulated particles according to the present invention further contain a surfactant (D).

Moreover, in the granulated particles according to the present invention, it is preferable that the poorly water soluble drug (A) and the diluent (B) be co-ground.

Furthermore, in the granulated particles according to the present invention, it is preferable that the diluent (B) be at least one powder selected from celluloses, saccharides, and starches.

Additionally, in the granulated particles according to the present invention, it is preferable that the proportion between the contents of the poorly water soluble drug (A) and the diluent (B) in the granulated particles in total and the content of the water soluble- or water swellable polymer compound (C) be 1/0.005 to 1/0.3 in terms of mass ratio.

Moreover, in the granulated particles according to the present invention, it is preferable that the poorly water soluble drug (A) be a nonsteroidal anti-inflammatory drug.

In addition, a second aspect according to the present invention is a tablet containing the granulated particles.

Moreover, a third aspect according to the present invention is a method for producing granulated particles characterized by including: co-grinding a poorly water soluble drug (A) and a diluent (B) so as to prepare a co-ground product having a volume average particle size within the range of 0.01 to 35 μm; and performing a wet granulation process while spraying an aqueous liquid, which contains a water soluble- or water swellable polymer compound (C) having a viscosity of less than 6.0 mPa·s in an aqueous solution thereof of 2% by mass at 20° C., to the co-ground product.

### EFFECTS OF THE INVENTION

According to the present invention, it will be possible to provide granulated particles that are excellent in terms of the drug release properties in the body, a tablet, and a method for producing the granulated particles.

### BEST MODE FOR CARRYING OUT THE INVENTION

### Granulated Particles

The granulated particles according to the present invention include a poorly water soluble drug (A) (hereinafter may be referred to as a component (A)) and a diluent (B) (hereinafter may be referred to as a component (B)) and in which a volume average particle size (hereinafter may simply be referred to as an “average particle size”) of the component (A) and the particles of the component (B) is within the range of 0.01 to 35 μm and a water soluble- or water swellable polymer compound (C) having a viscosity of less than 6.0 mPa·s in an aqueous solution thereof of 2% by mass at 20° C. is further contained.

It is preferable that the granulated particles according to the present invention further contain a surfactant (D) (hereinafter may be referred to as a component (D)) in addition to the abovementioned components (A) to (C).

The components (A) to (D) will be described in detail below.

### <Component (A)>

The granulated particles according to the present invention contain a poorly water soluble drug (A).

In the present invention, the phrase “poorly water soluble drug” refers to a drug having a water solubility of 0 to 30 mg/mL, preferably 0 to 10 mg/mL, at 20° C.

The type of the poorly water soluble drug (A) is not particularly limited. Specific examples thereof include nonsteroidal anti-inflammatory drugs such as ibuprofen, naproxen, ketoprofen, acetaminophen, indomethacin, bufexamac, aspirin, diclofenac, alclofenac, fenclofenac, etodolac, flurbiprofen, ketoprofen, mefenamic, meclofenamic, or piroxicam; sleeping pills/sedatives such as nitrazepam, triazolam, phenobarbital, or amibarbital; antiepileptic drugs such as phenyloin, metharbital, primidone, clonazepam, carbamazepine, or valproic acid; antidizziness drugs such as meclizine hydrochloride or dimenhydrinate; antidepressants such as imipramine, noxiptiline, or phenelzine; psychotropic drugs such as haloperidol, meprobamate, chlordiazepoxide, diazepam, oxazepam, or sulpiride; antispastic drugs such as papaverine, atropine, or etomidoline; cardiotonic agents such as digoxin, digitoxin, methyldigoxin, or ubidecarenone; antiarrhythmic drugs such as pindolol, ajmaline, or disopyramide; diuretics such as hydrochlorothiazide, spironolactone, triamterene, furosemide, or bumetanide; antihypertensive agents such as reserpine, dihydroergotoxine mesylate, prazosin hydrochloride, metoprolol, propranolol, or atenolol; coronary vasodilators such as nitroglycerin, isosorbide dinitrate, diltiazem, nifedipine, or dipyridamole; antitussive drugs such as noscapine, salbutamol, procaterol, tulobuterol, tranilast, or ketotifen; expectorants such as bromhexine hydrochloride or guaifenesin; ameliorants of cerebral circulation such as nicardipine or pinpocetin; antibiotics such as erythromycin, josamycin, chloramphenicol, tetracycline, rifampicin, or griseofulvin; antihistamines such as diphenhydramine, promethazine, mequitazine, or clemastine fumarate; steroid drugs such as triamcinolone, dexamethasone, betamethasone, prednisolone, danazol, methyltestosterone, or chlormadinone acetate; vitamins such as vitamin A, vitamin D, vitamin E, vitamin K, or folic acid (vitamin M); therapeutic agents for digestive system disorders such as dimethicone, famotidine, cimetidine, nizatidine, metoclopramide, famotidine, omeprazole, sulpiride, trepibutone, or sucralfate; and other agents such as caffeine, dicoumarol, cinnarizine, clofibrate, gefarnate, probenecid, mercaptopurine, methotrexate, ursodesoxycholic acid, or dihydroergotamine mesylate.

Among the abovementioned examples, the component (A) is preferably a nonsteroidal anti-inflammatory drug since the effects of the present invention can be achieved particularly markedly.

These examples of the component (A) may be used alone or two or more kinds thereof may be combined for use.

The content of the component (A) in the granulated particles may be set to the effective dose in the respective poorly water soluble drugs. For example, the content of the component (A) in the granulated particles is preferably about 30 to 90% by mass and more preferably about 50 to 75% by mass.

### <Component (B)>

The granulated particles according to the present invention contain a diluent (B).

Although the diluent (B) is not particularly limited, it is preferable that the diluent (B) be at least one powder selected from celluloses, saccharides, and starches since the effects according to the present invention improve particularly.

Specifically, preferable examples of the cellulose powders include crystalline cellulose, powdered cellulose, carmellose, hydroxypropyl cellulose, hydroxypropyl methylcellulose, low substituted hydroxypropyl cellulose, hydroxymethylcellulose, methylcellulose, and ethylcellulose. Among them, low substituted hydroxypropyl cellulose and crystalline cellulose are more preferable and low substituted hydroxypropyl cellulose is most preferable. (It should be noted that the above examples exclude the water soluble- or water swellable polymer compounds having a viscosity of less than 6.0 mPa·s in an aqueous solution thereof of 2% by mass at 20° C.).

In the present description, the phrase “low substituted” indicates that the mole substitution degree of a substituent (hydroxypropoxy group in the case of the aforementioned low substituted hydroxypropyl cellulose) is 5 to 16, preferably about 7 to 12.

Specifically, preferable examples of the powdered saccharides include monosaccharides, polysaccharides equal to or higher than disaccharides (e.g., sugars (such as granulated sugar), lactose, maltose, xylose, and isomerized lactose), sugar alcohols (e.g., PALATINITT, sorbitol, lactitol, erythritol, xylitol, saccharified products of reduced starch, maltitol, and mannitol), starch sirup, isomerized saccharides, oligosaccharides, sucrose, trehalose, and saccharified products of reduced starch (decomposed products of reduced starch).

Specifically, preferable examples of the powdered starches include starches such as corn starch, potato starch, wheat starch, or rice starch, and starch derivatives such as hydroxypropyl starch or partially gelatinized starch, and corn starch is more preferable than others.

Among the abovementioned examples, the diluent (B) is more preferably a cellulose powder or a powdered starch and particularly preferably a cellulose powder.

These examples of the component (B) may be used alone or two or more kinds thereof may be combined for use.

The content of the component (B) in the granulated particles is preferably 10 to 70% by mass and more preferably about 20 to 50% by mass. If the content of the component (B) is greater than or equal to the lower limit of the above range, when mixed with the aforementioned component (A), the attachment of the component (A) to the grinder is suppressed and the mixing efficiency and the grindability improve. On the other hand, if the content of the component (B) is less than or equal to the upper limit of the above range, the amount of the component (B) can be well balanced with those of other components so as to improve the effects according to the present invention.

In the granulated particles according to the present invention, it is preferable that the mixing proportion between the component (A) and the component (B), that is, the mass ratio ((A):(B)) is preferably 1:0.01 to 1:10, more preferably 1:0.05 to 1:5, and even more preferably 1:0.2 to 1:2. If the component (B) is greater than or equal to 0.01 relative to the component (A) in the above mass ratio ((A):(B)), the attachment of the component (A) to the grinder is suppressed and the mixing efficiency and the grindability improve when the two components are mixed. On the other hand, if the component (B) is less than or equal to 10 relative to the component (A) in the above mass ratio ((A):(B)), the effects according to the present invention improve.

In addition, it is preferable that the component (A) and the component (B) be co-ground. The surface area of the component (A) can further be increased when the component (A) and the component (B) are co-ground, thereby improving the effects according to the present invention. Moreover, the grinding of the poorly water soluble drugs that are originally not suited for grinding can also be carried out satisfactorily.

In the present invention, the volume average particle size of the particles of the component (A) and the particles of the component (B) is within the range of 0.01 to 35 μm, preferably within the range of 0.1 to 30 μm, and more preferably within the range of 1 to 25 μm. If the volume average particle size is greater than or equal to the lower limit of the above range, the surface area of the component (A) increases sufficiently, thereby improving the effects according to the present invention. Moreover, it will become easier to obtain uniform particles. On the other hand, if the volume average particle size is less than or equal to the upper limit of the above range, the effects according to the present invention or the granulability improves.

It should be noted that in the present invention, the phrase “volume average particle size” refers to the value measured by, for example, LS 230 (product name) manufactured by Beckman Coulter, Inc.

As the method for controlling the volume average particle size of the particles of the component (A) and the particles of the component (B) within the range of 0.01 to 35 μm, the component (A) and the component (B) may be ground separately and adjust the particle size of the respective components so as to achieve the above range of the volume average particle size, or the component (A) and the component (B) may be co-ground so as to adjust the particle size of the mixed particles in the co-ground product. Of these, the method to co-grind the component (A) and the component (B) so as to adjust the particle size of the mixed particles in the co-ground product is preferable since the effects due to the co-grinding process are achieved as described above.

### <Component (C)>

The granulated particles according to the present invention include the component (A) and the component (B) and further contains a water soluble- or water swellable polymer compound (C) having a viscosity of less than 6.0 mPa·s in an aqueous solution thereof of 2% by mass at 20° C. The effects according to the present invention are improved by further containing the component (C). In addition, the granulability also improves.

The viscosity of an aqueous solution of the component (C) of 2% by mass is less than 6.0 mPa·s at 20° C., preferably within the range of 1 to 5.5 mPa·s, more preferably within the range of 1.2 to 5.0 mPa·s, and particularly preferably within the range of 1.5 to 4.0 mPa·s. The effects according to the present invention are markedly improved when the viscosity is less than the upper limit of the above range.

Although it is not certain why such effects are achieved, it is considered that due to the use of the component (C), the rate at which the component (A) contained in the granulated particles is released in the body increases compared to the rates in conventional cases.

Note that the phrase “aqueous solution” used here includes the liquids in which a polymer compound is dissolved in water and the uniform liquids in which a polymer compound is swollen by absorbing water.

In addition, the “viscosity” in the present invention is measured by a Brookfield type viscometer (LVDVII+PRO (single cylindrical rotary viscometer manufactured by Brookfield Engineering Laboratories, Inc.) with a ULA spindle at 60 rpm) for 4 minutes at 20° C.

The component (C) is a water soluble- or water swellable polymer compound that satisfies the aforementioned viscosity conditions.

It should be noted that in the present description, the phrase “water soluble polymer compound” refers to a polymer compound having a water solubility of at least 1 mg/mL at 20° C., preferably greater than or equal to 10 mg/mL.

In addition, in the present description, the phrase “water swellable polymer compound” refers to a polymer compound that swells when mixed with water and forms a viscous liquid which is transparent, turbid, or suspended.

Examples of such water soluble or water swellable polymer compounds include celluloses such as carmellose, carmellose sodium, carmellose calcium, crosscarmellose sodium, hydroxypropyl cellulose, low substituted hydroxypropyl cellulose, hydroxypropyl methylcellulose, low substituted hydroxypropylmethylcellulose, or methyl cellulose; gum arabic, carboxyvinyl polymers, povidone, crosspovidone, polyvinyl alcohols, and polyacrylic acids. Of these, polyvinyl alcohols, hydroxypropyl cellulose, hydroxypropyl methylcellulose, and methyl cellulose are preferable. Among polyvinyl alcohols, those having a saponification degree of 96 mol % or less are more preferable.

These examples of the component (C) may be used alone or two or more kinds thereof may be combined for use.

The content of the component (C) in the granulated particles is preferably 0.1 to 20% by mass and more preferably about 1 to 15% by mass. If the content of the component (C) is greater than or equal to the lower limit of the above range, the effects according to the present invention are improved. On the other hand, if the content is less than or equal to the upper limit of the above range, the granulability improves.

In the granulated particles according to the present invention, the proportion between the contents of the component (A) and the component (B) in total in the granulated particles and the content (solid content) of the component (C), that is, the mass ratio ((A)+(B):(C)) is preferably 1:0.005 to 1:0.3 and more preferably 1:0.01 to 1:0.25. If the component (C) is greater than or equal to 0.005 in the above mass ratio ((A)+(B):(C)), the effects according to the present invention are improved. On the other hand, if the component (C) is less than or equal to 0.3 in the above mass ratio ((A)+(B):(C)), the granulability improves.

### <Component (D)>

It is preferable that the granulated particles according to the present invention further contain a surfactant (D) in addition to the components (A), (B), and (C). The effects according to the present invention are further improved by further containing the component (D).

The component (D) is not particularly limited and the surfactants such as nonionic surfactants, anionic surfactants, cationic surfactants, and amphoteric surfactants that are usually used in the oral formulations or the like can be used.

Examples of the nonionic surfactants include polyoxyethylene (2) alkyl ether, polyoxyethylene (9) alkyl ether, polyoxyethylene (21) alkyl ether, polyoxyethylene (25) alkyl ether, polyoxyethylene (5) alkyl phenyl ether, polyoxyethylene (10) alkyl phenyl ether, polyoxyethylene (15) alkyl phenyl ether, polyoxyethylene (10) polyoxypropylene (4) alkyl ether, polyoxyethylene (40) castor oil, polyoxyethylene (60) castor oil, polyoxyethylene (80) castor oil, polyoxyethylene (40) hardened castor oil, polyoxyethylene (60) hardened castor oil, polyoxyethylene (80) hardened castor oil, polyoxyethylene glycerin fatty acid ester, polyglycerin fatty acid ester, sorbitan fatty acid ester, polyoxyethylene (10) sorbitan fatty acid ester, polyoxyethylene (20) sorbitan fatty acid ester, polyoxyethylene (30) sorbitol fatty acid ester, polyoxyethylene (40) sorbitol fatty acid ester, polyoxyethylene (60) sorbitol fatty acid ester, polyoxyethylene (10) sterol, polyoxyethylene (20) sterol, polyoxyethylene (30) sterol, hydrogenated sterol, polyethylene glycol (1) fatty acid ester, polyethylene glycol (2) fatty acid ester, polyethylene glycol (4) fatty acid ester, polyethylene glycol (10) fatty acid ester, polyethylene glycol (25) fatty acid ester, polyethylene glycol (40) fatty acid ester, polyoxyethylene lanolin, polyoxyethylene lanolin alcohol, polyoxyethylene (6) beeswax derivatives, polyoxyethylene (20) beeswax derivatives, polyoxyethylene (5) alkylamine, polyoxyethylene (10) alkylamine, polyoxyethylene (15) alkylamine, polyoxyethylene (5) fatty acid amide, polyoxyethylene (10) fatty acid amide, polyoxyethylene (15) fatty acid amide, alkyldiethanolamine, alkyl glucoside, alkyl maltoside, alkylpolyglucoside, fatty acid sucrose ester, methyl glucoside ester, and methylglucamide.

Note that the values in the parentheses in the abovementioned examples of nonionic surfactants indicate the average number of moles of ethylene oxide (EO) added.

Examples of the anionic surfactants include alkyl ether carboxylates, N-acyl amino acid salts such as N-acyl sarcosine salts, N-acylglutamates, and N-acyl-N-methyl β-alanine salts, polyoxyethylene alkyl sulfates, α-olefin sulfonates, N-acyl-N-methyl taurates, alkyl sulfosuccinates, alkyl phosphates, and polyoxyethylene alkyl ether phosphates.

Examples of the cationic surfactants include N-acylaminoethyldiethylamine salts, and N-acylguanidine salts.

Examples of the amphoteric surfactants include lecithin derivatives such as soybean phospholipids, hydrogenated soybean phospholipids, egg yolk phospholipids, hydrogenated egg yolk phospholipids, or phosphatidyl choline; N-alkyldimethylamine oxide, N-alkyl-β-iminobipropionic acid salts, N-alkyldimethylbetaine, N-acyl-dimethylbetaine, N-acylamidopropyldimethyl betaine, 2-alkylimidazoline derivatives, N-alkylsulfobetaine glucamine, and N-alkylcarboxybetaine glucamine.

Among the above surfactants, nonionic surfactants, anionic surfactants, and amphoteric surfactants are preferable. Moreover, considering that they will be administered orally, nonionic surfactants are more preferable.

These examples of the component (D) may be used alone or two or more kinds thereof may be combined for use.

The content of the component (D) in the granulated particles is preferably 0.01 to 20% by mass and more preferably about 0.1 to 10% by mass. If the content of the component (D) is greater than or equal to the lower limit of the above range, the effects according to the present invention are improved. On the other hand, if the content is less than or equal to the upper limit of the above range, the granulability improves.

In the granulated particles according to the present invention, the proportion between the contents of the component (A) and the component (B) in total in the granulated particles and the content of the component (D), that is, the mass ratio ((A)+(B):(D)) is preferably 1:0.001 to 1:0.2 and more preferably 1:0.005 to 1:0.07. If the proportion of the component (D) is greater than or equal to the lower limit of the above range in the aforementioned mass ratio ((A)+(B):(D)), the effects according to the present invention are improved. On the other hand, if the proportion of the component (D) is less than or equal to the upper limit of the above range, the granulability improves.

In addition to the abovementioned components (A) to (D), the granulated particles according to the present invention may further contain any component that is usually used in medicinal preparations within a range, which does not adversely affect the effects according to the present invention.

The volume average particle size of the granulated particles according to the present invention is preferably within the range of 100 to 1,000 μm and more preferably within the range of 150 to 700 μm when tablets are prepared using the granulated particles.

In addition, the volume average particle size of the granulated particles is preferably within the range of 400 to 1,000 μm and more preferably within the range of 500 to 850 μm when granulated agents are prepared using the granulated particles.

Preferable examples of the methods for producing the granulated particles according to the present invention include methods that are similar to the method described later as one example of the methods for producing the granulated particles according to the present invention.

### <Tablet>

A tablet according to the present invention is made containing the granulated particles according to the present invention.

In addition to the granulated particles according to the present invention, the tablet may contain, if necessary, other materials, for example, a binder, a diluent such as a disintegrator, a lubricant, a perfume, or a flavoring (such as a sweetener or an acidulant).

Specifically, starch, gelatinized starch, sucrose, gelatin, gum arabic powder, methyl cellulose, carmellose, carmellose calcium, carmellose sodium, hydroxypropyl cellulose, hydroxypropyl methyl cellulose, polyvinylpyrrolidone, pullulan, dextrin, or the like can be used as a binder.

As a diluent, a disintegrator such as carmellose, carmellose calcium, carmellose sodium, crosscarmellose sodium, carboxymethyl cellulose calcium, or low substituted hydroxypropyl cellulose; lactose, corn starch, talc, crystalline cellulose (such as Avicel), powdered sugar, mannitol, light anhydrous silicic acid, calcium carbonate, L-cysteine, or the like can be used.

As a lubricant, magnesium stearate, calcium stearate, polyethylene glycol, talc, stearic acid, sucrose fatty acid esters, or the like can be used.

As a perfume, menthol, limonene, plant essential oils (such as peppermint oil, spearmint oil, lychee oil, orange oil, and lemon oil), or the like can be used.

As a sweetener, saccharine sodium, aspartame, stevia, dipotassium glycyrrhizinate, potassium acesulfame, thaumatin, sucralose, or the like can be used.

As an acidulant, for example, citric acid, tartaric acid, malic acid, succinic acid, fumaric acid, lactic acid, salts of these acids, or the like can be used.

The tablet according to the present invention can be produced, for example, by mixing the granulated particles according to the present invention with the abovementioned various materials where necessary and tabletting using a rotary tabletting machine such as Libra (product name) manufactured by Kikusui Seisakusho Ltd. and L-41 (product name) manufactured by Hata Iron Works Co., Ltd.

### <Method for Producing Granulated Particles>

The method according to the present invention for producing granulated particles is a production method in which a poorly water soluble drug (A) and a diluent (B) are co-ground so as to prepare a co-ground product having a volume average particle size within the range of 0.01 to 35 μm, and thereafter, performing a wet granulation process while spraying an aqueous liquid, which contains a water soluble- or water swellable polymer compound (C) having a viscosity of less than 6.0 mPa·s in an aqueous solution thereof of 2% by mass at 20° C., to the co-ground product.

One example of the method according to the present invention for producing granulated particles is described below and the step in which the co-ground product is prepared and the step in which a wet granulation process is performed are described individually.

[Step in which a Co-Ground Product is Prepared.]

In the present step, a poorly water soluble drug (A) and a diluent (B) are mixed and co-ground so as to prepare a co-ground product having a volume average particle size within the range of 0.01 to 35 μm.

In this process, the same materials as those listed above as the examples of the components (A) and (B) can be used as the poorly water soluble drug (A) and the diluent (B).

The co-grinding of the poorly water soluble drug (A) and the diluent (B) is carried out by, for example, using a grinder so that the co-ground product prepared by the co-grinding process will have a volume average particle size within the range of 0.01 to 35 μm.

The type of grinder used in the co-grinding process is not particularly limited and examples thereof include impact crushers such as a hammer mill, a sample mill, a disk mill, and a pin mill; a dry pulverizer such as a jet mill, a cylinder mill, and a roller mill. Among them, the impact crushers are preferable and the pin mill is more preferable.

[Step in which a Wet Granulation Process is Performed.]

In the present step, the granulated particles are produced by conducting a wet granulation process while spraying an aqueous liquid, which contains a water soluble- or water swellable polymer compound (C) having a viscosity of less than 6.0 mPa·s in an aqueous solution thereof of 2% by mass at 20° C., to the co-ground product prepared in the former step.

In this process, the same materials as those listed above as the examples of the component (C) can be used as the water soluble- or water swellable polymer compound (C).

It is preferable that the content of the water soluble- or water swellable polymer compound (C) in the aqueous liquid be within the range of 0.1 to 50% by mass. The granulability improves when the content is greater than or equal to the lower limit of the above range. On the other hand, the operational ease during the spraying of the aqueous liquid to the co-ground product or the like improves when the content is less than or equal to the upper limit of the above range.

In addition to the water soluble- or water swellable polymer compound (C) and water, the aforementioned surfactant (D) and/or other components such as ethanol and isopropyl alcohol may be added to the aqueous liquid.

Examples of the methods for performing a wet granulation process while spraying an aqueous liquid containing a water soluble- or water swellable polymer compound (C) to the co-ground product include a fluidized bed granulation process in which the granulation process is carried out by spraying the aqueous liquid containing the water soluble- or water swellable polymer compound (C) due to the use of a stirring fluidized-bed granulator such as Multiplex (product name) manufactured by Powrex Corporation or Spiral Flow (product name) manufactured by Freund Corporation; and a stirring granulation process in which the granulation process is carried out by spraying or adding dropwise the aqueous liquid containing the water soluble- or water swellable polymer compound (C) due to the use of a stirring granulator such as High Speed Mixer (product name) manufactured by Fukae Powtec Co., Ltd. and High Speed Stirring Granulator (product name) manufactured by Dalton Co., Ltd. to stir and forge, and thereafter, granulating by the use of an extrusion granulator such as Dome Gran (product name) manufactured by Dalton Co., Ltd. Of these, the fluidized bed granulation process is preferable in view of the improvements in the release properties of the poorly water soluble drug (A) in the body.

In the spraying of the aqueous liquid containing the water soluble- or water swellable polymer compound (C) to the co-ground product, it is preferable to adjust the spraying amount of the aqueous liquid so that the proportion between the contents of the poorly water soluble drug (A) and the diluent (B) in total in the granulated particles and the content (solid content) of the water soluble- or water swellable polymer compound (C) will be the mass ratio described earlier in the section for the granulated particles according to the present invention.

The volume average particle size of the granulated particles produced by such a production method is, when formed into a tablet or a granulated agent, preferably the same as the volume average particle size of the granulated particles, when formed into a tablet or a granulated agent, which is described earlier in the section for the granulated particles according to the present invention.

Note that a coating treatment using a coating agent may be conducted afterwards on the produced granulated particles where necessary in order to improve stability or the like.

As such a coating agent, it is preferable to select those which do not considerably impair the effects according to the present invention, that is, the release properties of the poorly water soluble drug (A) in the body, and it is more preferable to select water-soluble polymer compounds, saccharides, or the like.

Specific examples thereof include celluloses such as carmellose, hydroxypropyl cellulose, hydroxypropyl methylcellulose, low substituted hydroxypropyl cellulose, hydroxymethylcellulose, methylcellulose, and ethylcellulose; gum arabic, carboxyvinyl polymers, povidone, crosspovidone, polyvinyl alcohols, polyacrylic acids, monosaccharides, polysaccharides equal to or higher than disaccharides (e.g., sugars (such as granulated sugar), lactose, maltose, xylose, and isomerized lactose), sugar alcohols (e.g., PALATINITT, sorbitol, lactitol, erythritol, xylitol, saccharified products of reduced starch, maltitol, and mannitol), starch sirup, isomerized saccharides, oligosaccharides, sucrose, trehalose, and saccharified products of reduced starch (decomposed products of reduced starch).

These coating agents may be used alone or two or more kinds thereof may be combined for use.

The amount of coating agent to be used is preferably about 0.1 to 20 parts by mass with respect to 100 parts by mass of the granulated particles.

According to the present invention, it will be possible to provide granulated particles that are excellent in terms of the drug release properties in the body, a tablet, and a method for producing the granulated particles.

In addition, the granulated particles and the tablet provided by the present invention have satisfactory stability over time.

Moreover, the grindability is satisfactory in the present invention since it is unlikely that the component (A) will attach to the grinder when the components (A) and (B) are ground.

Furthermore, the present invention is excellent in terms of manufacturability since no problems occur in the handling after the grinding process.

Additionally, the present invention is also excellent in terms of granulability.

Moreover, according to the present invention, it will be possible to provide a medicinal preparation, preferably a solid chemical composition or the like such as a granular pharmaceutical composition or a tablet, which has high drug release properties in the body and contains a poorly water soluble drug having excellent immediate effects and efficacy.

### EXAMPLES

The present invention will be described in more detail below using Examples. However, the present invention is not limited to these Examples. In addition, the terms “parts” and “%” in the following Examples refer to a solid content without water and indicate “parts by mass” and “% by mass”, respectively, unless specifically stated otherwise.

### <Production of Granulated Particles>

The granulated particles in the respective Examples were produced due to the following production method using the poorly water soluble drugs, the diluents and the polymer compounds shown in Tables 1, 2, and 5, respectively.

It should be noted that the volume average particle size of the co-ground product prepared by the co-grinding process (the particles of a poorly water soluble drug and the particles of a diluent after the co-grinding process) and the volume average particle size of the obtained granulated particles A to T in the respective Examples are also shown in Tables 1, 2, and 5, respectively.

The volume average particle size was measured using LS 230 manufactured by Beckman Coulter, Inc (measurement conditions were: with a dry powder module and a vibrator 16, the auger was turned off, and the duration was 20 seconds).

### Example 1

60 parts of ibuprofen (having a volume average particle size of 70 μm before the co-grinding process) and 30 parts of low substituted hydroxypropyl cellulose (having a volume average particle size of 40 μm before the co-grinding process) were co-ground using a grinder (product name: Pin Mill) manufactured by Powrex Corporation. The volume average particle size of the co-ground product prepared by the co-grinding process (that is, the particles of ibuprofen and the particles of low substituted hydroxypropyl cellulose after the co-grinding process) was 12 μm. By conducting a fluidized bed granulation process while spraying an aqueous solution of 6% by mass of a polyvinyl alcohol (having a saponification degree of 87.5 mol %) that had a viscosity of 3.0 mPa·s in an aqueous solution thereof of 2% by mass at 20° C. to the obtained co-ground product using Spiral Flow (product name of a stirring fluidized-bed granulator manufactured by Freund Corporation), the granulated particles A containing 10 parts of the polyvinyl alcohol were produced. The volume average particle size of the obtained granulated particles A was about 350 μm.

### Example 2

The granulated particles B were produced in the same manner as that of Example 1 according to the composition shown in Table 1. The volume average particle size of the co-ground product prepared by the co-grinding process (the particles of a poorly water soluble drug and the particles of a diluent after the co-grinding process) was 11 μm. In addition, the volume average particle size of the obtained granulated particles B was about 350 μm.

### Example 3

The granulated particles C were produced in the same manner as that of Example 1 according to the composition shown in Table 1. The volume average particle size of the co-ground product prepared by the co-grinding process (the particles of a poorly water soluble drug and the particles of a diluent after the co-grinding process) was 15 μm. In addition, the volume average particle size of the obtained granulated particles C was about 350 μm.

### Examples 4 to 9

The granulated particles 0 to T were respectively produced in the same manner as that of Example 1 according to the composition shown in Tables 1 and 2.

The volume average particle size of the co-ground product prepared by the co-grinding process (the particles of a poorly water soluble drug and the particles of a diluent after the co-grinding process) and the volume average particle size of the obtained granulated particles O to T are also shown in Tables 1 and 2, respectively.

### Comparative Example 1

The granulated particles D were produced in the same manner as that of Example 1 according to the composition shown in Table 1. The volume average particle size of the co-ground product prepared by the co-grinding process (the particles of a poorly water soluble drug and the particles of a diluent after the co-grinding process) was 20 μm. In addition, the volume average particle size of the obtained granulated particles D was about 350 μm.

### Comparative Example 2

The granulated particles E were produced in the same manner as that of Example 1 according to the composition shown in Table 1. It should be noted that the volume average particle size of the co-ground product prepared by the co-grinding process (the particles of a poorly water soluble drug and the particles of a diluent after the co-grinding process) was 40 μm. In addition, the volume average particle size of the obtained granulated particles E was about 500 μm.

### Comparative Example 3

The granulated particles F were produced in the same manner as that of Example 1 according to the composition shown in Table 1. The volume average particle size of the co-ground product prepared by the co-grinding process (the particles of a poorly water soluble drug and the particles of a diluent after the co-grinding process) was 40 μm. In addition, the volume average particle size of the obtained granulated particles F was about 350 μm.

### Comparative Examples 4 and 5

The granulated particles M and N were respectively produced in the same manner as that of Example 1 according to the composition shown in Table 2.

The volume average particle size of the co-ground product prepared by the co-grinding process (the particles of a poorly water soluble drug and the particles of a diluent after the co-grinding process) and the volume average particle size of the obtained granulated particles M and N are also shown in Table 2, respectively.

### Examples 20 to 25

The granulated particles G to L were respectively produced in the same manner as that of Example 1 according to the composition shown in Table 5.

The volume average particle size of the co-ground product prepared by the co-grinding process (the particles of a poorly water soluble drug and the particles of a diluent after the co-grinding process) and the volume average particle size of the obtained granulated particles G to L are also shown in Table 5, respectively.

### <Production of Tablet>

### Examples 10 to 19 and 26 to 37, and Comparative Examples 6 to 10

The materials for tablets shown in Tables 3, 4, 6, and 7, respectively, were mixed and the resulting mixtures were tableted using Libra (product name of a rotary tabletting machine manufactured by Kikusui Seisakusho Ltd.) to obtain the tablets of the respective Examples.

<Evaluation of Drug Release Properties from Granulated Particles and Tablets>

The drug release properties from the granulated particles or the tablets were evaluated in accordance with a paddle method defined as a dissolution test in Japanese Pharmacopoeia.

In terms of the test conditions, 59.5 g of sodium acetate and 33.2 mL of acetic acid were added and dissolved in 20 L of purified water to prepare a test solution and the pH of the test solution was adjusted to 4.5.

The test was carried out by charging granulated particles or a tablet into the test solution, setting the paddle rotational frequency at 50 rpm, collecting 10 mL of the test solution at each of the predetermined time points while stirring, and measuring the elution rate (amount of drug released with respect to the initial amount of poorly water soluble drug (set amount of poorly water soluble drug contained in granulated particles)) by high performance liquid chromatography.

The time at which the elution rate of 90% by mass was achieved was set as the release time (minute) and the release time less than or equal to 15 minutes was determined as satisfactory in terms of drug release properties in the body.

In addition, the abovementioned test was carried out with the granulated particles and the tablets immediately after the production thereof and those after being preserved at 40° C. for 6 months, respectively. Evaluation results are shown in Tables 1 to 7 (evaluation results of granulated particles in Tables 1, 2, and 5, and evaluation results of tablets in Tables 3, 4, 6, and 7).

TABLE 1

| | | Examples | | | | | | |
|---|---|---|---|---|---|---|---|---|
| | | 1 | 2 | 3 | 4 | 5 | 6 | 7 |
| Granulated particles | | A | B | C | O | P | Q | R |
| (A) | Ibuprofen (average particle size before | 60 | | 60 | 60 | 60 | 60 | 60 |
| | grinding: 70 μm) | | | | | | | |
| | Aspirin (average particle size before grinding: | | 40 | | | | | |
| | 80 μm) | | | | | | | |
| | Acetaminophen (average particle size before | | 20 | | | | | |
| | grinding: 150 μm) | | | | | | | |
| | Bromhexine hydrochloride (average particle | | | 2 | | | | |
| | size before grinding: 60 μm) | | | | | | | |
| | Anhydrous caffeine (average particle size | | | 5 | | | | |
| | before grinding: 150 μm) | | | | | | | |
| (B) | Low substituted hydroxypropyl cellulose | 30 | 30 | 23 | 30 | 30 | 30 | 30 |
| | (average particle size before grinding: 40 μm) | | | | | | | |
| (C) | Polyvinyl alcohol (2% by mass, viscosity at | 10 | | | | | | |
| | 20° C.: 3.0 mPa · s) | | | | | | | |
| | Polyvinyl alcohol (2% by mass, viscosity at | | | | | | | |
| | 20° C.: 15.0 mPa · s) | | | | | | | |
| | Polyvinylpyrrolidone (2% by mass, viscosity | | | | 10 | | | |
| | at 20° C.: 1.5 mPa · s) | | | | | | | |
| | Hydroxypropyl cellulose (2% by mass, | | 10 | 10 | | | 10 | 10 |
| | viscosity at 20° C.: 2.5 mPa · s) | | | | | | | |
| | Hydroxypropyl cellulose (2% by mass, | | | | | 10 | | |
| | viscosity at 20° C.: 5.5 mPa · s) | | | | | | | |
| | Hydroxypropyl cellulose (2% by mass, | | | | | | | |
| | viscosity at 20° C.: 8.0 mPa · s) | | | | | | | |
| | Hydroxypropyl methylcellulose (2% by mass, | | | | | | | |
| | viscosity at 20° C.: 15.0 mPa · s) | | | | | | | |

| Total (parts by mass) | | 100 | 100 | 100 | 100 | 100 | 100 | 100 |
|---|---|---|---|---|---|---|---|---|
| Average particle size of co-ground product (μm) | | 12 | 11 | 15 | 12 | 16 | 1 | 28 |
| Average particle size of granulated particles (μm) | | 350 | 350 | 350 | 350 | 350 | 350 | 350 |
| Release time | Immediately after production | 11 | 11 | 12 | 10 | 12 | 10 | 13 |
| (min) | After preservation at 40° C. for 6 | 11 | 12 | 12 | 11 | 12 | 11 | 12 |
| | months | | | | | | | |

TABLE 2

| | | Examples | | Comparative Examples | | | | |
|---|---|---|---|---|---|---|---|---|
| | | 8 | 9 | 1 | 2 | 3 | 4 | 5 |
| Granulated particles | | S | T | D | E | F | M | N |
| (A) | Ibuprofen (average particle size before | 60 | 60 | 60 | 60 | 60 | 60 | 60 |
| | grinding: 70 μm) | | | | | | | |
| | Aspirin (average particle size before grinding: | | | | | | | |
| | 80 μm) | | | | | | | |
| | Acetaminophen (average particle size before | | | | | | | |
| | grinding: 150 μm) | | | | | | | |
| | Bromhexine hydrochloride (average particle | | 2 | | | | | |
| | size before grinding: 60 μm) | | | | | | | |
| | Anhydrous caffeine (average particle size | | 5 | | | | | |
| | before grinding: 150 μm) | | | | | | | |
| (B) | Low substituted hydroxypropyl cellulose | 25 | 20 | 30 | 30 | 30 | 30 | 30 |
| | (average particle size before grinding: 40 μm) | | | | | | | |
| (C) | Polyvinyl alcohol (2% by mass, viscosity at | 10 | | | | | | |
| | 20° C.: 3.0 mPa · s) | | | | | | | |
| | Polyvinyl alcohol (2% by mass, viscosity at | | | | | | 10 | |
| | 20° C.: 15.0 mPa · s) | | | | | | | |
| | Polyvinylpyrrolidone (2% by mass, viscosity | | | | | | | |
| | at 20° C.: 1.5 mPa · s) | | | | | | | |
| | Hydroxypropyl cellulose (2% by mass, | | 10 | | | 10 | | |
| | viscosity at 20° C.: 2.5 mPa · s) | | | | | | | |
| | Hydroxypropyl cellulose (2% by mass, | | | | | | | |
| | viscosity at 20° C.: 5.5 mPa · s) | | | | | | | |
| | Hydroxypropyl cellulose (2% by mass, | | | 10 | 10 | | | |
| | viscosity at 20° C.: 8.0 mPa · s) | | | | | | | |
| | Hydroxypropyl methylcellulose (2% by mass, | | | | | | | 10 |
| | viscosity at 20° C.: 15.0 mPa · s) | | | | | | | |
| (D) | Polyoxyethylene sorbitan fatty acid ester | 5 | | | | | | |
| | (product name: Polysorbate 80; average | | | | | | | |
| | number of moles of ethylene oxide (EO) | | | | | | | |
| | added: 20 moles) | | | | | | | |
| | Polyoxyethylene hardened castor oil (average | | 3 | | | | | |
| | number of moles of EO added: 60 moles) | | | | | | | |

| Total (parts by mass) | | 100 | 100 | 100 | 100 | 100 | 100 | 100 |
|---|---|---|---|---|---|---|---|---|
| Average particle size of co-ground product (μm) | | 16 | 16 | 20 | 40 | 40 | 20 | 20 |
| Average particle size of granulated particles (μm) | | 350 | 350 | 350 | 500 | 350 | 350 | 350 |
| Release time | Immediately after production | 8 | 9 | 19 | 28 | 25 | 23 | 24 |
| (min) | After preservation at 40° C. for 6 | 9 | 10 | 20 | 30 | 24 | 22 | 23 |
| | months | | | | | | | |

TABLE 3

| | | Examples | | | | | | | |
|---|---|---|---|---|---|---|---|---|---|
| Tablet | | 10 | 11 | 12 | 13 | 14 | 15 | 16 | 17 |
| Granulated particles A | | 70 | | | | | | | |
| Granulated particles B | | | 70 | | | | | | |
| Granulated particles C | | | | 70 | | | | | |
| Granulated particles O | | | | | 70 | | | | |
| Granulated particles P | | | | | | 70 | | | |
| Granulated particles Q | | | | | | | 70 | | |
| Granulated particles R | | | | | | | | 70 | 50 |
| Lactose | | 10 | 10 | 10 | 10 | 10 | 10 | 10 | 8 |
| Crystalline cellulose | | 16 | 16 | 16 | 16 | 16 | 16 | 16 | 13 |
| Crosscarmellose sodium | | 3 | 3 | 3 | 3 | 3 | 3 | 3 | 3 |
| Magnesium stearate | | 1 | 1 | 1 | 1 | 1 | 1 | 1 | 1 |
| Magnesium oxide | | | | | | | | | 25 |
| Total (parts by mass) | | 100 | 100 | 100 | 100 | 100 | 100 | 100 | 100 |
| Release time | Immediately after | 12 | 12 | 11 | 11 | 13 | 11 | 13 | 8 |
| (min) | production | | | | | | | | |
| | After preservation at 40° C. | 11 | 12 | 12 | 11 | 12 | 12 | 13 | 8 |
| | for 6 months | | | | | | | | |

TABLE 4

| | | Examples | | Comparative Examples | | | | |
|---|---|---|---|---|---|---|---|---|
| Tablet | | 18 | 19 | 6 | 7 | 8 | 9 | 10 |
| Granulated particles S | | 70 | | | | | | |
| Granulated particles T | | | 70 | | | | | |
| Granulated particles D | | | | 70 | | | | |
| Granulated particles E | | | | | 70 | | | |
| Granulated particles F | | | | | | 70 | | |
| Granulated particles M | | | | | | | 70 | |
| Granulated particles N | | | | | | | | 70 |
| Lactose | | 10 | 10 | 10 | 10 | 10 | 10 | 10 |
| Crystalline cellulose | | 16 | 16 | 16 | 16 | 16 | 16 | 16 |
| Crosscarmellose sodium | | 3 | 3 | 3 | 3 | 3 | 3 | 3 |
| Magnesium stearate | | 1 | 1 | 1 | 1 | 1 | 1 | 1 |
| Total (parts by mass) | | 100 | 100 | 100 | 100 | 100 | 100 | 100 |
| Release time | Immediately after production | 10 | 10 | 21 | 30 | 29 | 28 | 29 |
| (min) | After preservation at 40° C. for 6 | 10 | 11 | 22 | 31 | 30 | 27 | 29 |
| | months | | | | | | | |

TABLE 5

| | | Examples | | | | | |
|---|---|---|---|---|---|---|---|
| | | 20 | 21 | 22 | 23 | 24 | 25 |
| Granulated particles | | G | H | I | J | K | L |
| (A) | Ibuprofen (average particle size before grinding: 70 μm) | 60 | 60 | 60 | | | |
| | Aspirin (average particle size before grinding: 80 μm) | | | | 50 | | |
| | Famotidine (average particle size before grinding: | | | | 10 | | |
| | 100 μm) | | | | | | |
| | Cimetidine (average particle size before grinding: | | 2 | 2 | | 70 | |
| | 100 μm) | | | | | | |
| | Sucralfate (average particle size before grinding: | 10 | 8 | 8 | 8 | | 70 |
| | 120 μm) | | | | | | |
| (B) | Low substituted hydroxypropyl cellulose (average | 21 | | | 22 | 21 | 21 |
| | particle size before grinding: 40 μm) | | | | | | |
| | Crystalline cellulose (average particle size before | | 20 | | | | |
| | grinding: 50 μm) | | | | | | |
| | Corn starch (average particle size before grinding: | | | 20 | | | |
| | 40 μm) | | | | | | |
| (C) | Polyvinyl alcohol (2% by mass, viscosity at 20° C.: | 9 | | | | | |
| | 3.0 mPa · s) | | | | | | |
| | Hydroxypropyl cellulose (2% by mass, viscosity at | | 10 | | | 9 | |
| | 20° C.: 4.0 mPa · s) | | | | | | |
| | Hydroxypropyl methylcellulose (2% by mass, | | | 10 | | | 9 |
| | viscosity at 20° C.: 4.0 mPa · s) | | | | | | |
| | Methylcellulose (2% by mass, viscosity at 20° C.: 4.0 mPa · s) | | | | 10 | | |

| Total (parts by mass) | | 100 | 100 | 100 | 100 | 100 | 100 |
|---|---|---|---|---|---|---|---|
| Average particle size of co-ground product (μm) | | 15 | 15 | 15 | 10 | 12 | 15 |
| Average particle size of granulated particles (μm) | | 350 | 350 | 350 | 350 | 350 | 350 |
| Release time | Immediately after production | 10 | 11 | 12 | 13 | 10 | 12 |
| (min) | After preservation at 40° C. for 6 months | 10 | 12 | 11 | 13 | 11 | 13 |

(Components (B) and (C) used in Examples)
Low substituted hydroxypropyl cellulose: LH-21 manufactured by Shin-Etsu Chemical Co., Ltd. (average particle size before grinding: 40 μm, mole substitution degree: 10.8); Polyvinyl alcohol (2% by mass, viscosity at 20° C.: 3.0 mPa·s): Gohsenol EG-05 manufactured by Nippon Synthetic Chemical Industry Co., Ltd.;
Polyvinyl alcohol (2% by mass, viscosity at 20° C.: 15.0 mPa·s): Gohsenol EG-30 manufactured by Nippon Synthetic Chemical Industry Co., Ltd.;
Polyvinylpyrrolidone (2% by mass, viscosity at 20° C.: 1.5 mPa·s): Plasdone K-25 manufactured by ISP Technologies Inc.;
Hydroxypropyl cellulose (2% by mass, viscosity at 20° C.: 2.5 mPa·s): NISSO HPC-SSL manufactured by Nippon Soda Co., Ltd.;
Hydroxypropyl cellulose (2% by mass, viscosity at 20° C.: 5.5 mPa·s): NISSO HPC-SL manufactured by Nippon Soda Co., Ltd.;
Hydroxypropyl cellulose (2% by mass, viscosity at 20° C.: 8.0 mPa·s): NISSO HPC-L manufactured by Nippon Soda Co., Ltd.;
Hydroxypropyl methylcellulose (2% by mass, viscosity at 20° C.: 15.0 mPa·s): Metolose SM-15 manufactured by Shin-Etsu Chemical Co., Ltd.

Viscosity measurements of the component (C) were made under the following conditions.

Rotational viscometer: LVDVII+PRO (monocylindrical rotary viscometer manufactured by Brookfield Engineering Laboratories, Inc.);
Spindle No. ULA;
Measuring vessel: tall beaker (500 mL);
Amount of liquid measured: about 450 mL;
Measuring temperature: 20° C.;
Rotational frequency: 60 rpm;
Measuring time: 4 minutes

TABLE 6

| | | Examples | | | | | |
|---|---|---|---|---|---|---|---|
| Tablet | | 26 | 27 | 28 | 29 | 30 | 31 |
| Granulated particles G | | 80 | | | | | |
| Granulated particles H | | | 80 | | | | |
| Granulated particles I | | | | 80 | | | |
| Granulated particles J | | | | | 80 | | |
| Granulated particles K | | | | | | 80 | |
| Granulated particles L | | | | | | | 80 |
| Corn starch | | 10 | 10 | 10 | 10 | 10 | 10 |
| Low substituted | | 9 | 9 | 9 | 9 | 9 | 9 |
| hydroxypropyl cellulose | | | | | | | |
| Calcium stearate | | 1 | 1 | 1 | 1 | 1 | 1 |
| Total (parts by mass) | | 100 | 100 | 100 | 100 | 100 | 100 |
| Release time | Immediately | 9 | 12 | 13 | 13 | 10 | 12 |
| (min) | after | | | | | | |
| | production | | | | | | |
| | After | 10 | 14 | 14 | 13 | 11 | 13 |
| | preservation | | | | | | |
| | at 40° C. for | | | | | | |
| | 6 months | | | | | | |

TABLE 7

| | | Examples | | | | | |
|---|---|---|---|---|---|---|---|
| Tablet | | 32 | 33 | 34 | 35 | 36 | 37 |
| Granulated particles B | | 70 | 70 | 70 | | | 60 |
| Granulated particles L | | | | | 70 | 70 | 10 |
| Corn starch | | 10 | 10 | 10 | 10 | 10 | 10 |
| Crystalline cellulose | | 5 | 5 | 5 | 5 | 5 | 5 |
| Low substituted | | 9 | 9 | 9 | 9 | 9 | 9 |
| hydroxypropyl cellulose | | | | | | | |
| Hydrous silicon dioxide | | 5 | 3 | 3 | 3 | 3 | 3 |
| Crosspovidone | | | 2 | | 2 | | |
| Crosscarmellose sodium | | | | 2 | | 2 | 2 |
| Calcium stearate | | 1 | 1 | 1 | 1 | 1 | 1 |
| Total (parts by mass) | | 100 | 100 | 100 | 100 | 100 | 100 |
| Release time | Immediately | 12 | 10 | 10 | 14 | 14 | 14 |
| (min) | after | | | | | | |
| | production | | | | | | |
| | After | 13 | 11 | 10 | 15 | 15 | 15 |
| | preservation | | | | | | |
| | at 40° C. for | | | | | | |
| | 6 months | | | | | | |

It was verified from the results shown in Tables 1, 2, and 5 that the granulated particles of Examples 1 to 9 and 20 to 25 according to the present invention were excellent in terms of drug release properties in the body compared to the granulated particles of Comparative Examples 1 to 5.

In addition, it was verified from the results shown in Tables 3, 4, 6, and 7 that the tablets of Examples 10 to 19 and 26 to 37 according to the present invention were excellent in terms of drug release properties in the body compared to the tablets of Comparative Examples 6 to 10.

### INDUSTRIAL APPLICABILITY

According to the present invention, it will be possible to provide granulated particles that are excellent in terms of the drug release properties in the body, a tablet, and a method for producing the granulated particles.

1. Granulated particles comprising:
a poorly water soluble drug (A); and
a diluent (B),
wherein a volume average particle size of particles of the poorly water soluble drug (A) and particles of the diluent (B) is within a range of 0.01 to 35 μm, and
a water soluble- or water swellable polymer compound (C) having a viscosity of less than 6.0 mPa·s in an aqueous solution thereof of 2% by mass at 20° C. is further contained.

2. The granulated particles according to claim 1, further comprising a surfactant (D).

3. The granulated particles according to claim 1, wherein the poorly water soluble drug (A) and the diluent (B) are co-ground.

4. The granulated particles according to claim 1, wherein the diluent (B) is at least one powder selected from celluloses, saccharides, and starches.

5. The granulated particles according to claim 1,
wherein a proportion between contents of the poorly water soluble drug (A) and the diluent (B) in total in the granulated particles and a content of the water soluble- or water swellable polymer compound (C) is 1:0.005 to 1:0.3 in terms of mass ratio.

6. The granulated particles according to claim 1, wherein the poorly water soluble drug (A) is a nonsteroidal anti-inflammatory drug.

7. A tablet comprising the granulated particles of claim 1.

8. A method for producing granulated particles comprising:
co-grinding a poorly water soluble drug (A) and a diluent (B) so as to prepare a co-ground product having a volume average particle size within a range of 0.01 to 35 μm; and
performing a wet granulation process while spraying an aqueous liquid, which comprises a water soluble- or water swellable polymer compound (C) having a viscosity of less than 6.0 mPa·s in an aqueous solution thereof of 2% by mass at 20° C., to the co-ground product.
